(19) United States
(12) Patent Application Publication
(10) Pub. No.: US 2007/0031507 A1
(43) Pub. Date: Feb. 8, 2007

(54) Bactericide for MRSA

(76) Inventors: Nobuji Koyama, Aomori (JP); Shinichi Okuda, Miyagi (JP); Koji Sasaya, Kanagawa (JP); Tomonaga Yoshida, Aomori (JP)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/544,699

(22) Filed: Oct. 6, 2006

Related U.S. Application Data
(63) Continuation of application No. 10/491,456, filed on Aug. 17, 2004.

(30) Foreign Application Priority Data
Oct. 2, 2001 (JP) 2001-306597

(51) Int. Cl. A61K 35/56; A61K 33/10

(52) U.S. Cl. 424/547; 424/687

(57) ABSTRACT

A bactericide for MRSA and a method for making the bactericide are disclosed. The bactericide is characterized in that it comprises a product prepared by pulverizing a shell having a crystalline structure of a calcite structure of calcium carbonate. The bactericide allows a wide variety of articles to be sterilized by the use of one type of bactericide, which leads to simplification of the management of chemicals for use in sterilization and learning of methods for using them and thus to saving in work.

### CROSS REFERENCE TO RELATED APPLICATIONS

The present application is a continuation application under U.S.C. §120 of U.S. application Ser. No. 10/491,456, entitled BACTERICIDE FOR MRSA and filed Aug. 17, 2004, which claims the priority benefit of a §371 National Phase Filing of PCT/JP02/10241, internationally filed on Oct. 2, 2002, and further claims priority to Japanese Application No. 2001-306597 filed on Oct. 2, 2001, the whole of which are each hereby incorporated by reference herein.

### STATEMENT REGARDING FEDERALLY SPONSORED RESEARCH OR DEVELOPMENT

N/A

### BACKGROUND OF THE INVENTION

There are possibilities of invasion of MRSA (methicillin resistant Staphylococcus aureus) through MRSA carriers and infected patients in hospital in recent years, and emergence of new MRSA infection in the hospital from MRSA infection patients and through facilities used by the infected patients such as beds in the hospital room is also suspected. While various prophylactic measures have been taken against MRSA, frequent emergence of MRSA infection diseases in hospital is a large problem yet.

MRSA invades into hospital through the MRSA carriers and MRSA infection patients, or normal bacterium is denatured into MRSA in the hospital by administration of antibiotics. Onset of MRSA diseases is considered to be ascribed to direct or indirect infection among patients or from the patient to medical workers. Particularly, MRSA is transmitted via fingers of the medical workers, used tools and medical devices in most cases. Since resistance to antibiotics of MRSA tends to be enhanced by using the antibiotics, use of the antibiotics to the infected patients should be restricted to remedies of MRSA infection diseases, and use of the antibiotics for prevention of infection and for disinfection of MRSA in non-infected patients should be avoided. Accordingly, a variety of chemicals have been used for disinfection of patients and medical workers by washing their hands, and for sterilization of various medical devices and facilities used in the hospital in order to suppress transmission of the bacteria as much as possible.

However, it is a current situation that the chemicals used for disinfection and sterilization are different depending on the object of disinfection and sterilization. Management of these chemicals is quite complicated to cause troubles in essential medical works.

For solving the problem above, the inventors of the invention have noticed that a powder obtained by calcinating and pulverizing shells of, for example, scallops that have been disposed as wastes has a bactericidal effect inherent in natural products due to its alkaline property. An object of the invention is to provide a bactericide that is able to disinfect reliably and effectively by using only one kind of the bactericide even when a variety of objects are to be disinfected. Another object of the invention is to provide a method for simplifying management and learning of chemicals used for disinfection in the current situation, thereby saving the labor of the medical workers.

### BRIEF SUMMARY OF THE INVENTION

The present invention relates to bactericide for MRSA.

### DETAILED DESCRIPTION OF THE INVENTION

The invention has been performed by taking the problems above, and to solve the problems above, provides a bactericide for MRSA comprising a product manufactured by pulverizing shells having a calcite crystal structure of calcium carbonate.

In the invention, the above-described product can be a mixture of a calcium carbonate powder as a shell powder having a calcite crystal structure of calcium carbonate containing porous particles, and a calcium oxide powder prepared by calcinating the calcium carbonate powder.

The invention for solving the problems above further provides the bactericide for MRSA being an aqueous solution prepared by dissolving the above-described product in water.

Best Mode for Carrying Out the Invention

The invention will be described in detail hereinafter based on the embodiments of the invention.

Shells such as scallop shells having a calcite crystal structure of calcium carbonate at least at the main portion of the shell are prepared for obtaining the bactericide for MRSA of the invention. While various shells are available for this object, bivalves, particularly scallop has the calcite crystal structure at most part of the shell.

For example, scallops are featured in quick motion in its wild life for escaping from predators (such as starfish), as expressed as “swimming in the sea” by strongly spouting seawater by an opening/closing action of the shell. Scallop has a large ligament of scallop for enabling this action while the shell itself is relatively thin and lightweight with a high mechanical strength. Leaflets of the calcite crystal structure of calcium carbonate are formed at the inner side of the shell as a structure, while platelets of the calcite crystal structure of calcium carbonate are formed in the inner layer of the shell (core layer in the direction of thickness of the shell). Since the main part (except the surface layer and hinge of the shell) comprises the structures of the inner face side and inner layer of the shell, the shell has a high mechanical strength while it is thin and lightweight.

The main part of the scallop shell comprises, as described above, a leaflet structure of the calcite crystal structure of calcium carbonate (like kenzan in Japanese—a spiked plate used for flower arrangement) at the inner face side, and a platelet structure of the calcite crystal structure of calcium carbonate (a structure comprising plate structures of needle-like structure aligned in the same direction and laminated in layers like a plywood, and the direction of the needle-like crystals is different among the layers) in the inner layer of the shell. Accordingly, as described below, the particles obtained by pulverizing the shell of scallop as a bivalve become porous by leaving the calcite structure. On the contrary, since the calcium carbonate crystals of shells in other shapes are spread over the surface of the shell to exhibit pearly luster, the shell does not become porous when granulated.

The scallop shell was used as a material for obtaining the bactericide for MRSA of the invention. The scallop shell disposed as a useless waste after removing the ligament of scallop can be effectively utilized in the invention. The shells disposed as useless are collected and dried in the sunlight to harden them. The hardened shell after drying in the sunlight is pulverized into particles with a particle diameter of about 200 μm. The pulverizing method itself is not particularly restricted, and any pulverizing machines available may be used. The calcium carbonate powder comprising porous particles is obtained as described above.

Subsequently, a part of the calcium carbonate powder comprising the porous particles as described above is heated in a rotary furnace at a temperature of about 1050° C. for about several hours to obtain a calcium oxide powder. The calcium oxide powder obtained as described above and the calcium carbonate powder are mixed to obtain a product that is used as the bactericide for MRSA. The bactericide for MRSA can be also obtained by dispersing the product in water.

The calcium carbonate powder in the bactericide for MRSA is the powder of the scallop shell as a natural product, which has antibacterial activities common in various natural products. The natural products are in contrast to chemically synthesized substances, and the shell of caltivated scallop also belongs to the natural product. The calcium oxide powder is alkaline, and shows a high antibacterial activity. Accordingly, this bactericide for MRSA is able to suppress proliferation of bacteria by exhibiting an antibacterial activity inherent in the natural products and antibacterial activity due to its alkaline property. Disinfection is possible by adhering the powdery bactericide for MRSA to various tools in hospital and interior goods in the hospital room, or by embedding the tools in this powdery bactericide. Disinfection is also possible by immersing a part of the tools in an aqueous solution of this bactericide for MRSA or by coating the bactericide on the tool, and by spraying the bactericide in the hospital room. Accordingly, the object to be treated with the bactericide for MRSA is not particularly restricted.

The bactericide for MRSA was obtained from the product as a mixture of the porous particles of the calcium carbonate powder as a shell powder having the calcite crystal structure of calcium carbonate and the calcium oxide powder prepared by calcinating the calcium carbonate powder, and the aqueous solution of the product was also used as the bactericide for MRSA. However, the invention is not restricted to these preparations. For example, the product may comprise only the calcium carbonate powder, or only the calcium oxide powder. Furthermore, the shell as the starting material of the product is not restricted to the scallop shell, and any shells as natural products may be used.

Antibacterial tests were then performed. The relation between the cultivation time (minute) of MRSA in a cultivation medium and the survival ratio (%) of the bacteria was measured in the test shown in Table 1. The cultivation media of MRSA were physiological saline (medium 1), physiological saline containing the bactericide for MRSA of the invention (medium 1) and physiological saline containing a reagent NaOH (medium 3). The degree of dilution of both the bactericide for MRSA and the reagent (NaOH) was 10⁻³, and pH of both solutions was 12.7.

TABLE 1

| | Cultivation Time (minutes) | | | |
|---|---|---|---|---|
| | 0 | 5 | 15 | 30 |
| Medium 1 | 100% | — | — | 88.2% |
| Medium 2 | 100% | 4.34% | 0.022% | 0.00036% |
| Medium 3 | 100% | — | Measurement was impossible | 0.0057% |
| | | | due to a large survival ratio | |

It could be confirmed from the results in Table 1 that the bactericide for MRSA of the invention has a good bactericidal action. Accordingly, the bactericide for MRSA of the invention is effective for disinfection of patients, and disinfection of facilities in surgical laboratories and the hands of workers in charge of surgery. The bactericide can be also used for disinfection treatment in an infected area out of the hospital, and disinfection treatment of medical wastes. In addition, the bactericide of the invention is effective for disinfection of bedroom articles such as bed clothes and mat covers in the hospital room, disinfection of clothes such as white clothes and footwear in the hospital, and disinfection of implements used in the hospital room.

The bactericide of the invention is also effective for disinfection of MRSA in kitchen facilities in hospital and schools, kitchen facilities in public buildings and restaurants, food production factories and food processing factories.

### INDUSTRIAL APPLICABILITY

According to the invention as described above, the bactericide for MRSA of the invention has good disinfection action available for various disinfection purposes. Accordingly, since disinfection of MRSA is possible by using only the bactericide of the invention without using a variety of chemicals depending on the object of disinfection, efficiency of the disinfection work is improved. Since the bactericide for MRSA of the invention comprises materials of natural origin without containing any chemically synthesized substances, it is an advantage of the bactericide of the invention that no adverse effect is caused for workers handling the bactericide.

Since shells that have been disposed as needless wastes are effectively utilized, the invention can contribute for reducing the mass of wastes. The invention exerts an excellent practical effect since the bactericide for MRSA has a simple constitution without carrying out any complicated manufacturing process.

What is claimed is:

1. A method for preparing a bactericide for MRSA comprising pulverizing shells having a calcite crystal structure of calcium carbonate, whereby a product containing a powder of porous calcium carbonate particles is produced.

2. The method of claim 1, further comprising calcinating a first portion of said powder of porous calcium carbonate particles, whereby a calcium oxide powder is produced, and forming a mixture of said calcium oxide powder and a second portion of said powder of porous calcium carbonate particles.

3. The method of claim 1, further comprising dispersing said product in water.

4. The method of claim 2, further comprising dispersing said mixture in water.

5. The method of claim 1, wherein said shells are scallop shells.

6. The method of claim 2, wherein said shells are scallop shells.

7. The method of claim 3, wherein said shells are scallop shells.

8. The method of claim 4, wherein said shells are scallop shells.
